(19) United States
(12) Patent Application Publication
(10) Pub. No.: US 2008/0262745 A1
(43) Pub. Date: Oct. 23, 2008

(54) Method for Determining Insulin Sensitivity and Glucose Absorption

(76) Inventor: David Charles Polidori, Rancho Santa Fe, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/106,479

(22) Filed: Apr. 21, 2008

Related U.S. Application Data
(60) Provisional application No. 60/912,998, filed on Apr. 20, 2007.
(60) Provisional application No. 60/980,230, filed on Oct. 16, 2007.

(51) Int. Cl. G01N 33/50

(52) U.S. Cl. 702/19

(57) ABSTRACT

The present invention encompasses a model-based method for determining insulin sensitivity and glucose absorption from oral glucose tolerance tests or mixed meals. The present invention has several advantages over current methods. The technique requires about four to six blood samples taken over about two to three hours following glucose ingestion and is therefore applicable to large-scale clinical trials. The analysis involves a reduced version of the classical minimal model, a method for describing glucose absorption using only two parameters, and an integral approach enabling the parameters to be obtained using simple algebra. The present method robustly identifies differences in insulin sensitivity in different patient types as well as improvements in insulin sensitivity arising from pharmaceutic therapy. In addition, insulin sensitivity measurements obtained with the present method are highly correlated with results from hyperinsulinemic clamps (r²>0.8). This method is therefore a practical and robust method for determining insulin sensitivity under physiologic conditions.

### CROSS REFERENCE TO RELATED APPLICATIONS

This application claims priority of U.S. Provisional Patent Application Ser. Nos. 60/912,998 and 60/980,230 filed Apr. 20, 2007 and Oct. 16, 2007, respectively and entitled “A Method for Determining Insulin Sensitivity and Glucose Absorption.”

### BACKGROUND OF THE INVENTION

Insulin resistance is a characteristic feature of a number of metabolic diseases including obesity, type 2 diabetes and the metabolic syndrome. Several approaches have previously been developed to determine insulin sensitivity based on fasting measurements, glucose tolerance tests, or euglycemic, hyperinsulinemic clamps. See, e.g., 5122362.

There is great interest in measuring insulin sensitivity in patients to quantify the improvement in insulin sensitivity achieved with different therapies, identify insulin sensitivity in individuals to provide further insight into their pathophysiology and to determine optimal treatment approaches and identify changes in insulin sensitivity as early markers of disease progression. In addition, there is considerable interest in determining insulin sensitivity in preclinical research, for similar reasons.

Recently, several methods for determining insulin sensitivity from oral glucose tolerance tests (OGTTs) or meal tests have been proposed. One method involves a two-step procedure in which tracers are used to determine the rate of glucose absorption and then the classical minimal model analysis is used to determine insulin sensitivity. The experimental difficulty associated with this method makes it impractical for large studies. Dalla Man et al. (2005a) have developed an approach for simultaneously identifying parameters describing glucose absorption and insulin sensitivity using seven or more blood samples from meal challenges or OGTTs. This method was validated against multiple tracer methods in non-diabetic subjects and results were well correlated with results from hyperinsulinemic clamps. Dalla Man et al. (2005b). However, this method requires at least seven blood samples and there are as many parameters to be identified as data points collected. In addition, sophisticated modeling software is required and there is no guarantee that a unique, optimal solution will be found. Caumo et al. (2000) derived an index of insulin sensitivity by assuming the rate of glucose absorption closely follows the plasma glucose concentrations during a meal and integrating the equations over a period of time long enough to ensure that both glucose concentrations and insulin action have returned to basal values.

Other, more empiric methods, of determining insulin sensitivity from OGTTs have also been proposed. Stumvoll et al. (2000) empirically obtained an insulin sensitivity measure based on glucose and insulin measurements during an OGTT that was correlated with the glucose infusion rate during a hyperinsulinemic clamp. Matsuda et al. (1999) developed a composite insulin sensitivity measure based on both fasting and mean values of glucose and insulin and showed that this measure was correlated with results from a hyperinsulinemic clamp. Hansen et al. (2007) empirically determined measures of insulin sensitivity from OGTT that were correlated with S_{I} measured by IVGTT. Mari et al. (2001) have also developed a measure of insulin sensitivity (OGIS) based on fitting differential equations describing glucose kinetics at a single time point and then empirically determining several unknown quantities in order to match hyperinsulinemic clamp results. Although the OGIS approach was originally model-based, by fitting the differential equation at a single time point, much of the information in the glucose and insulin profiles is ignored.

### SUMMARY OF THE INVENTION

The present invention encompasses a model-based method for determining insulin sensitivity and glucose absorption from oral glucose tolerance tests or mixed meals. The present invention has several advantages over current methods. The technique requires about four to six blood samples taken over about two to three hours following glucose ingestion and is therefore applicable to large-scale clinical trials. The analysis involves a reduced version of the classical minimal model, a method for describing glucose absorption using only two parameters, and an integral approach enabling the parameters to be obtained using simple algebra. The present method robustly identifies differences in insulin sensitivity in different patient types as well as improvements in insulin sensitivity arising from pharmaceutic therapy. In addition, insulin sensitivity measurements obtained with the present method are highly correlated with results from hyperinsulinemic clamps (r²>0.8). This method is therefore a practical and robust method for determining insulin sensitivity under physiologic conditions.

The present invention encompasses a method of determining insulin sensitivity from an oral glucose tolerance test or mixed meals by measuring blood glucose levels and analyzing the results of the measurement with

$$\begin{matrix}\frac{d G}{d t} = \frac{{R}_{a}^{exo} ( t )}{{V}_{G}} - {S}_{I} ( G \cdot {I}_{i} - {G}_{basal} \cdot {I}_{basal} ) & ( 1 ) \\ \frac{d {I}_{i}}{d t} = \frac{1}{τ} ( {I}_{plasma} - {I}_{i} ) & ( 2 )\end{matrix}$$

where
G(t) is the plasma glucose concentration in mg/dl
Rₐ^{exo}(t) is the rate of appearance of exogenous glucose into the plasma (from meals or injections/infusions) in mg/min
V_{G} is the distribution volume of glucose in dl
S_{I} is insulin sensitivity in 1/min/(μU/ml)
Iᵢ(t) is the interstitial insulin concentration, in μU/ml (In addition to a time delay between plasma and interstitial insulin concentrations, interstitial concentrations are also lower than plasma concentrations, even in steady state conditions; this difference is not accounted for in these equations. Thus, the proper interpretation of Iᵢ(t) is as the actual interstitial insulin concentration at time t multiplied by the ratio of basal plasma insulin/basal interstitial insulin)
G_{basal} is the basal plasma glucose concentration in mg/dl
I_{basal} is the basal plasma insulin concentration μU/ml
τ is the time constant associated with transfer of insulin from plasma to interstitial fluid, in min
Iₚₗₐₛₘₐ is the plasma insulin concentration, in μU/ml.

The results can be obtained from any number of samples, preferably at least about four to six samples are obtained. Results obtained from four samples are sufficient to determine insulin sensitivity. The results can be obtained during any time period, preferably the time period is of about two to four hours. Results obtained from a two hour time period are sufficient to determine insulin sensitivity.

The method can be used to determine the effect of therapy on insulin sensitivity. The therapy can be any known in the art including, without limitation, pharmaceutical, nutritional or behavioral.

The method has numerous applications, for instance, it can be used in preclinical studies to determine the effect of a therapy; as a prognostic to assess a patient's risk of developing a disease or syndrome such as diabetes or metabolic syndrome; to monitor and/or adjust patient treatment; or in conjunction with automated insulin delivery.

### BRIEF DESCRIPTION OF THE DRAWINGS

FIG. 1: Plasma glucose and insulin for two of the groups in the Rosiglitazone study. Mean (±s.e.m.) values for glucose and insulin before treatment are shown in A and B. The response to treatment for the 8 diabetic subjects treated with Rosiglitazone is shown in C and D.

FIG. 2: Correlation between S_{I} obtained from the OGTT using the S_{I}ARₐA method and GIR during the hyperinsulinemic clamps. A: Results for 0.5 mU/kg/min insulin infusion. B: Results for 1.5 mU/kg/min insulin infusion. The individual points are the values for each of the 18 subjects in the study.

FIG. 3: Sensitivity of the S_{I} values obtained from the S_{I}ARₐA method to the assumptions about glucose absorption. All 11 data points were used in this analysis. A: Results using the present invention. B: Results using the classical minimal model. Each line depicts the results for one of the 18 subjects. The different assumptions are described in the text.

FIG. 4: Representative profiles for the two different absorption assumptions. A: Constant absorption assumption. B: Decreasing absorption assumption. Both cases are drawn with T_{end}=210 min.

### DETAILED DESCRIPTION OF THE INVENTION

Insulin resistance plays a major role in several metabolic diseases, including diabetes, obesity, and hypertension. Reaven (1988). As such, determining insulin sensitivity for patients is often of considerable clinical interest. The two most accepted methods for determining insulin sensitivity are the euglycemic, hyperglycemic clamp (DeFronzo et al. (1979)) and the frequently sampled intravenous glucose tolerance test (FSIVGTT) with minimal model analysis. Bergman (1989). Both of these methods deliver glucose in a non-physiologic manner and therefore provide assessments of insulin sensitivity under artificial conditions. In addition, the clamp procedure is experimentally difficult and costly and the FSIVGTT requires frequent blood sampling and modeling analysis. As a result, there is considerable interest in developing simpler means of determining insulin sensitivity under physiologic conditions.

The simplest methods that have been developed to determine insulin sensitivity use only a single blood sample obtained during fasting conditions. The two most popular of these indices are HOMA-IR (Matthews et al. (1985)) and QUICKI (Katz et al. (2000)), which are combinations of fasting glucose and insulin concentrations. Although these indices are easy to obtain, several reports show that they are not very well correlated with other measures of insulin sensitivity. Emoto et al. (1999); Brun et al. (2000); Yokoyama et al. (2003); and Cutfield et al. (2003).

The present invention provides a new model-based method for determining insulin sensitivity from OGTTs or mixed meals. This approach includes a reduced form of the classical minimal model of glucose metabolism, a simpler approach to determining the parameters based on integrating the equations, and some assumptions allowing the rate of glucose absorption (Rₐ) to be described using only two parameters. This approach has several advantages over previous methods. First, the method can be performed using as few as about four to six blood samples taken over a two-hour period. Second, the model contains only three parameters that are identified from the data: insulin sensitivity (S_{I}) and two parameters describing the glucose absorption profile. Third, the present approach allows the parameter values to be obtained using only simple algebra and a unique solution is guaranteed. Finally, results from the reduced mathematical model are less sensitive to the assumptions about glucose absorption than those for the classical minimal model, and a statistical criterion shows that the reduced model is preferred over the classical minimal model in these applications. Results are presented using the present method showing that it can be used to identify differences in insulin sensitivity in different patient types, can determine the change in insulin sensitivity in response to pharmaceutic therapy, and S_{I} determined by this approach is highly correlated with insulin sensitivity measured by hyperinsulinemic clamps. We have named this method S_{I}ARₐA for: S_{I} And Rₐ via Algebra.

The following examples are provided to illustrate but not limit the invention. All references cited herein are hereby incorporated herein by reference.

### EXAMPLES

### Example 1

### Research Design and Methods

The S_{I}ARₐA method involves the use of a reduced version of the classical minimal model of glucose metabolism, a method for describing the rate of absorption of glucose during the meal using only two parameters, and an integral approach to finding the optimal parameter values. A brief summary of these three components is provided below; the details are described subsequently.

### Mathematical Model

The following mathematical model, which is described in more detail below, is used to describe glucose kinetics during the OGTT.

$$\begin{matrix}\frac{d G}{d t} = \frac{{R}_{a}^{exo} ( t )}{{V}_{G}} - {S}_{I} ( G \cdot {I}_{i} - {G}_{basal} \cdot {I}_{basal} ) & ( 1 ) \\ \frac{d {I}_{i}}{d t} = \frac{1}{τ} ( {I}_{plasma} - {I}_{i} ) & ( 2 )\end{matrix}$$

where
G(t) is the plasma glucose concentration in mg/dl
Rₐ^{exo}(t) is the rate of appearance of exogenous glucose into the plasma (from meals or injections/infusions) in mg/min
V_{G} is the distribution volume of glucose in dl
S_{I} is insulin sensitivity in 1/min/(μU/ml)
Iᵢ(t) is the interstitial insulin concentration, in μU/ml (In addition to a time delay between plasma and interstitial insulin concentrations, interstitial concentrations are also lower than plasma concentrations, even in steady state conditions. This difference is not accounted for in these equations. Thus, the proper interpretation of Iᵢ(t) is as the actual interstitial insulin concentration at time t multiplied by the ratio of basal plasma insulin/basal interstitial insulin)
G_{basal} is the basal plasma glucose concentration in mg/dl
I_{basal} is the basal plasma insulin concentration μU/ml
τ is the time constant associated with transfer of insulin from plasma to interstitial fluid, in min
Iₚₗₐₛₘₐ is the plasma insulin concentration, in μU/ml
   Describing Rate of Appearance of Glucose from the Meal Using Two Parameters

Studies of gastric emptying and glucose absorption suggest that the rate of appearance of glucose following a meal generally follows one of two profiles. Dalla Man et al. (2005); Hunt et al. (1985); Brener et al. (1983); and Schirra et al. (1996). In one profile, glucose is absorbed at a fairly constant rate during the postprandial period. In the other profile, glucose absorption decreases exponentially. An approach for describing each of these profiles using only two parameters: f₃₀ (the fraction of ingested glucose absorbed in the first 30 minutes) and T_{end} (the time by which the majority of glucose in the meal has been absorbed) is described below.

### Integral Approach to Determining Parameter Values

Equation 2 can be integrated to obtain Iᵢ(t) from measured plasma insulin values. Equation 1 can then be integrated over each of the time intervals where glucose and insulin are measured to yield the following set of linear algebraic equations for the optimal parameter values:

$$( \begin{matrix}G ( {t}_{2} ) - G ( {t}_{1} ) \\ G ( {t}_{3} ) - G ( {t}_{2} ) + 0.9 {a}_{23} \\ G ( {t}_{4} ) - G ( {t}_{3} ) + 0.9 {a}_{34} \\ ⋮\end{matrix} ) = ( \begin{matrix}- {Φ}_{12} & {G}_{meal} / {V}_{G} \\ - {Φ}_{23} & {a}_{23} \\ - {Φ}_{34} & {a}_{34} \\ ⋮ & ⋮\end{matrix} ) ( \begin{matrix}{S}_{I} \\ {f}_{30}\end{matrix} ) \qquad ( 3 )$$

where
t₁, t₂, . . . are times at which glucose and insulin are measured
G(tᵢ) is plasma glucose at time tᵢ
Φᵢⱼ are integrals computed from glucose and insulin measured at tᵢ and tⱼ
Gₘₑₐₗ is the amount of glucose ingested, in mg
αᵢⱼ are measures of glucose absorption between tᵢ and tⱼ that are calculated from meal size and assumed absorption profiles
   The unique values of S_{I} and f₃₀ that minimize the squared error in Equation 3 can be easily obtained from standard algebraic methods. A final step is to perform a 1-dimensional optimization to find the value of T_{end} which gives the best fit between the model and the data.

### Statistical Analyses

Results are presented as mean ±s.e.m. Comparisons between groups were made using t-tests. Akaike's information criteria corrected (AICc) was used to compare models. Burnham et al. (2002).

### Data

Data from two previously performed clinical trials were used to validate the method.

### Study 1: Rosiglitazone Study

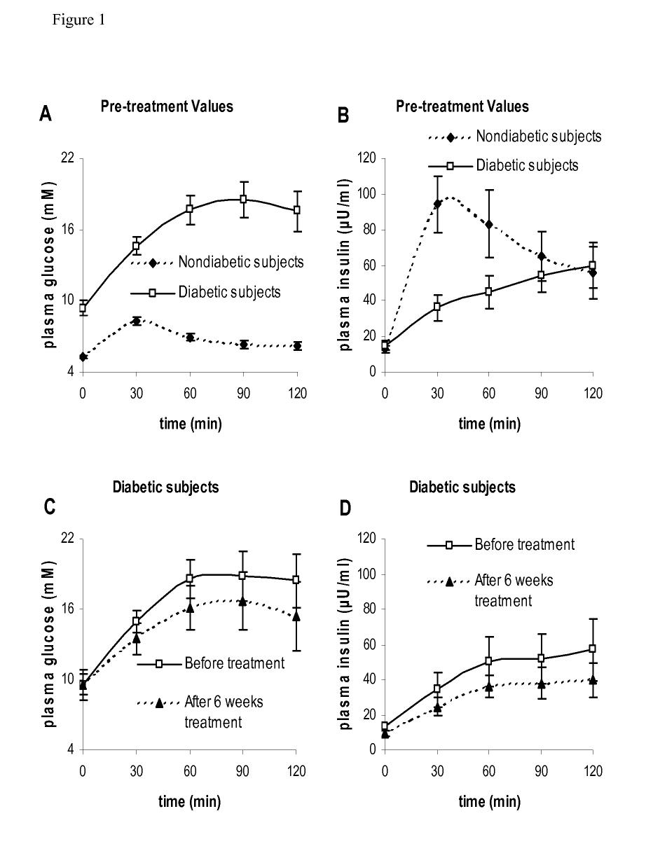

27 subjects (15 healthy, 12 with type 2 diabetes) were enrolled in a study attempting to identify markers of insulin sensitivity. Diabetic patients were taken off any oral medications during a two-week placebo run-in period and patients who had recently been using thiazolidinediones, NSAIDs, or were insulin-dependent were excluded. After the run-in period, 13 subjects (9 healthy, 4 with diabetes) received placebo for 6 weeks while the other 14 subjects (6 healthy, 8 with diabetes) received rosiglitazone (4 mg bid). Subjects were given OGTTs at the end of the run-in period and again after six weeks of treatment. Glucose and insulin were measured at t=0, 30, 60, 90, and 120 minutes. Glucose was measured using glucose oxidase method (Hitachi 747), and insulin was measured by RIA (Medigenix Diagnostics). The pre-treatment characteristics of the subjects are shown in Table 1 and glucose and insulin profiles during the OGTTs are shown in FIG. 1.

TABLE 1
Baseline characteristics (mean and (range)) for the subjects in the trials.

| | Study 1 | | Study 2 |
|---|---|---|---|
| | Healthy | Type 2 diabetic | Type 2 diabetic |
| | volunteers | subjects | subjects |
| | (n = 15) | (n = 12) | (n = 18) |
| Age (years) | 25.1 (18-51) | 51.3 (22-73) | 56 (43-63) |
| BMI (kg/m²) | 24.7 (18-34) | 29.3 (21-41) | 28.8 (22-36) |
| HbA1c (%) | 4.7 (4.2-5.2) | 6.4 (4.5-8.4) | 7.2 (5.4-9.6) |
| Fasting glucose (mM) | 5.2 (4.8-6.6) | 9.4 (5.3-12.5) | 10.1 (7.9-12.3) |

### Study 2: OGTT and Hyperinsulinemic Clamp Study

Eighteen type 2 diabetic patients enrolled in a clinical trial received both an OGTT and a three-step hyperinsulinemic, euglycemic clamp prior to any treatment. Baseline characteristics of the patients are shown in Table 1. The first two hours of the clamp was a run-in period where insulin was infused at 0.25 mU/kg/min. During the next two hours (Clamp 1), insulin was infused at 0.5 mU/kg/min and for the final two hours (Clamp 2), insulin was infused at 1.5 mU/kg/min. During Clamps 1 and 2, the average glucose infusion rate (GIR) during the last thirty minutes was calculated as a measure of insulin sensitivity. During the OGTT, glucose and insulin were measured at t=0, 5, 10, 15, 30, 60, 90, 120, 150, 180, and 240 minutes after glucose ingestion. Glucose was measured using glucose oxidase method (Super G Ambulance), and insulin was measured using RIA (by IKFE).

### Results

### Study 1

### Fit of Reduced Model to Data

The average value of r² for the fit in Equation 3 was 0.96 for the 54 cases tested, suggesting that the proposed model provides an excellent approximation to the actual data.

### Differences in S_{I} in Different Patient Types

S_{I} was calculated for each of the 15 nondiabetic subjects and the 12 diabetic subjects before treatment. Insulin sensitivity was significantly higher in the nondiabetic subjects than in the diabetic subjects (S_{I}=10.1±1.3 vs. 4.9±0.7 (10⁻⁴/Min/(μU/ml)), p<0.001).

Increase in S_{I} with Treatment

The analysis shows a significant increase in S_{I} in the 14 rosiglitazone-treated subjects (S_{I}=14.2±2.4 after treatment vs. 6.9±1.3 before treatment (10⁻⁴/min/(μU/ml)), p<0.05) and no increase in S_{I} in the 13 subjects in the placebo group (S_{I}=8.1±1.3 at week 6 vs. 8.6±1.4 at week 0). The approximate doubling of S_{I} with rosiglitazone treatment is similar to reports from hyperinsulinemic clamp studies with Rosiglitazone. Carey et al. (2002); and Mayerson et al. (2002).

### Variability in S_{I} for Repeated Measurements

The 13 individuals in the placebo group received two OGTTs, six weeks apart. The average variability in the calculated S_{I} values between measurements for these individuals was 35±7%.

### Glucose Absorption Parameters

The amount of glucose absorbed in the first 30 minutes estimated by the S_{I}ARₐA method was higher in the diabetic subjects than in the nondiabetic subjects (f₃₀=0.17±0.01 vs. 0.12±0.01, p<0.01). T_{end} showed a trend towards being faster in the diabetic subjects, although this difference was not statistically significant (T_{end}=187±11 min in the diabetic subjects vs. 211±15 min in the nondiabetic subjects, p=0.2). Rosiglitazone treatment did not lead to any significant differences in f₃₀ or T_{end}. The best fit to the data was obtained using the constant glucose absorption profile for 43 of the 54 cases tested.

### Study 2

### Results Using all 11 Blood Samples

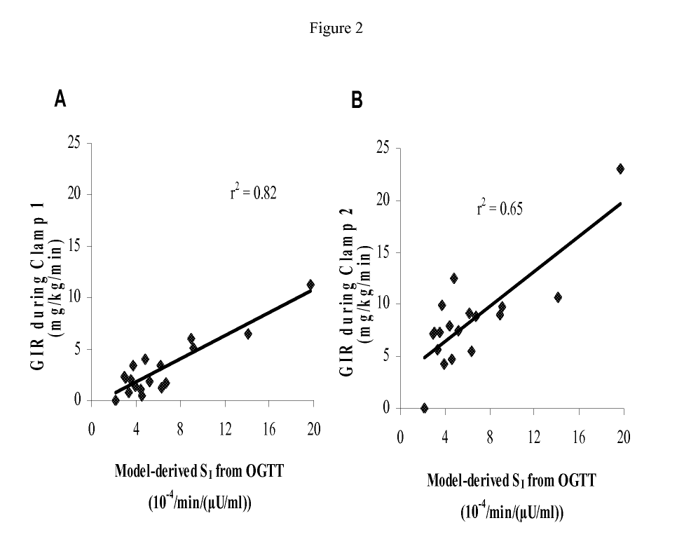

For each of the 18 subjects, S_{I} was determined from the OGTT using the S_{I}ARₐA method and compared with the GIR during Clamp 1 and Clamp 2. An excellent correlation was obtained for both clamps as shown in FIG. 2. As expected, because results from the S_{I}ARₐA method are obtained using OGTT data where insulin concentrations remain in the physiologic range, the correlation is higher with results from Clamp 1 than Clamp 2.

### Results Using Only 5 Blood Samples

To test how well the analysis works with a reduced number of blood samples, the same analysis was done using only five of the blood samples taken over two hours (t=0, 30, 60, 90, and 120 min). Nearly identical correlations between model-derived S_{I} and results from the clamps were obtained using only 5 data points obtained over 2 hours as when using all 11 points taken over 4 hours (Table 2). In Table 2, the correlation between different measures of insulin sensitivity and hyperinsulinemic-clamp derived insulin sensitivity. Results for the S_{I}ARₐA method are compared to results for other methods that can be applied using only the five data points collected at t=0, 30, 60, 90, and 120 min.

TABLE 2

| Insulin sensitivity method | GIR during Clamp 1 | GIR during Clamp 2 |
|---|---|---|
| S_{I}ARₐA (using 11 samples) | r² = 0.82 | r² = 0.65 |
| S_{I}ARₐA (using 5 samples) | r² = 0.85 | r² = 0.59 |
| HOMA-IR (4) | r² = 0.40 | r² = 0.37 |
| QUICKI (5) | r² = 0.57 | r² = 0.39 |
| ISIₑₛₜ (13) | r² = 0.60 | r² = 0.43 |
| ISI_{comp} (14) | r² = 0.70 | r² = 0.49 |
| BIGTT-S_{I\|0-30-120} (15) | r² = 0.78 | r² = 0.56 |
| BIGTT-S_{I\|0-60-120} (15) | r² = 0.75 | r² = 0.49 |
| OGIS (16) | r² = 0.55 | r² = 0.47 |

Comparison of Results with Other Insulin Sensitivity Methods

Several previously proposed insulin sensitivity measures can also be easily calculated using 5 or fewer data points collected over 2 hours after an OGTT. Insulin sensitivity values obtained using these methods were calculated and correlated with the hyperinsulinemic clamp results. Results from the S_{I}ARₐA method were more highly correlated with clamp results than any of the other methods (Table 2).

Sensitivity of Results to Modeling Assumptions and Comparison of Reduced Model with Classical Minimal Model

As described below, the analysis can be done using the classical minimal model rather than the reduced model proposed here. When using the classical minimal model an additional parameter for glucose sensitivity, S_{G}, is also identified.

### Details of Mathematical Model

### Description of Model

Circulating glucose concentrations can be described by the following equation:

$${V}_{G} \frac{d G}{d t} = {R}_{a}^{exo} ( t ) + {R}_{a}^{end} ( G , {I}_{i} ) - {R}_{d} ( G , {I}_{i} ) \qquad ( A1 )$$

where V_{G} is the distribution volume of glucose (dl), G is the plasma glucose concentration (mg/dl), Iᵢ is the interstitial insulin concentration (μU/ml), Rₐ^{exo} and Rₐ^{end} are the rate of appearance of glucose from exogenous sources (e.g., meals or infusions) and endogenous sources (liver, kidney), respectively (in mg/min), and R_{d} is the rate of disposal of glucose (in mg/min), and t is time (in min).

The term Rₐ^{end}(G,Iᵢ)−R_{d}(G,Iᵢ) is in general a nonlinear function of glucose and insulin that is not completely known. We do know that in the basal, overnight fasted state Rₐ^{end}(G_{basal},I_{basal})−R_{d}(G_{basal},I_{basal})=0. In addition, we know that both glucose and insulin act to decrease endogenous glucose output and to increase glucose disposal. Therefore, the following approximation is proposed

Rₐ^{end}(G,Iᵢ)−R_{d}(G,Iᵢ)≈−S_{I}(G·Iᵢ−G_{basal}·I_{basal}) (A2)

This approximation satisfies both of the above conditions using only a single parameter, S_{I}, which has units of 1/min/(μU/ml). Substituting Equation (A2) into Equation (A1) gives Equation 1 in the main text. In addition, the Equation 2 from the main text is used to account for the time delay associated with insulin transfer between plasma and interstitial fluid.
Comparison with Classical Minimal Model

The classical minimal model (Bergman (1989)) is

$$\begin{matrix}\frac{d G}{d t} = \frac{{R}_{a}^{exo} ( t )}{{V}_{G}} - XG - {p}_{1} ( G - {G}_{basal} ) & ( A3 ) \\ \frac{d X}{d t} = - {p}_{2} X + {p}_{3} ( {I}_{plasma} - {I}_{basal} ) & ( A4 )\end{matrix}$$

By defining Iᵢ=(p₂/p₃) X+I_{basal}, S_{I}=p₃/p₂, and S_{G}=p₁, and τ=1/P₂, Equation A4 compares to Equation 2 and Equation A3 becomes

$$\frac{d G}{d t} = \frac{{R}_{a}^{exo} ( t )}{{V}_{G}} - {S}_{I} ( G \cdot {I}_{i} - {G}_{basal} \cdot {I}_{basal} ) - ( {S}_{G} - {S}_{I} \cdot {I}_{basal} ) ( G - {G}_{basal} ) \qquad ( A5 )$$

Note that Equation A5 compares to Equation 1, with the additional (S_{G}−S_{I}·I_{basal})(G−G_{basal}) term in the classical minimal model. The difference S_{G}−S_{I}·I_{basal} is referred to as GEZI (Glucose Effectiveness at Zero Insulin) and it has been reported that it is difficult to accurately estimate GEZI from IVGTT data, particularly when there are a limited number of samples taken. Sakamoto et al. (1997). The S_{I}ARₐA method is also applicable using the classical minimal model (as described below), but in the analyses of OGTT data, better results were obtained with the reduced model described by Equations 1-2.

Describing Rate of Appearance from Meals Using Two Parameters

Results from gastric emptying studies and tracer studies identifying rates of glucose absorption often show one of two different profiles for gastric emptying and/or glucose absorption. In one of the profiles, there is an initial rapid phase of gastric emptying followed by a regulated phase in which approximately 2-4 kcal/min are emptied by the stomach. Hunt et al. (1985); and Brener et al. (1983). The rate of glucose absorption also rises rapidly, then reaches a steady value until decaying rapidly as the meal is nearly fully absorbed. In the other profile, the rate of gastric emptying and glucose absorption both rise rapidly, then continue to decrease in what appears to be an exponential fashion. Schirra et al. (1996). Examples of both profiles can be seen in (Dalla Man et al. (2005a)), where the OGTT resembles the constant profile described here and the mixed meal resembles the decreasing profile. The different profiles may be due to the type of food consumed (e.g., solid vs. liquid) and/or individual variability. Because it is not known in advance which profile will be most appropriate for a given patient/meal challenge, approximate both profiles and use the model to select the one that gives the best fit.

Although gastric emptying profiles like those described above have often been reported, there are also individuals who have significantly delayed gastric emptying. This includes patients with gastroparesis and can occur with some treatments such as exenatide. Therefore, the analysis does not assume that gastric emptying is initially rapid; rather, the model is used to determine the fraction of glucose in the meal that is absorbed in the first 30 minutes (parameter f₃₀). Doing this enables the model to be used to identify changes in gastric emptying and/or nutrient absorption in addition to insulin sensitivity.

Next, several assumptions enable computation of all of the integrals involving Rₐ^{end} as functions of f₃₀. First, assume that by the end of the postprandial period, 90% of the ingested glucose has been absorbed. This value was also used by Dalla Man et al. (2004) and is within the range of values reported in the literature (70-100%). Caumo et al. (2000); and Livesey et al. (1998). The time at which 90% of the meal has been absorbed is denoted T_{end} and this parameter value is obtained by solving a one-dimensional optimization problem, as described later.

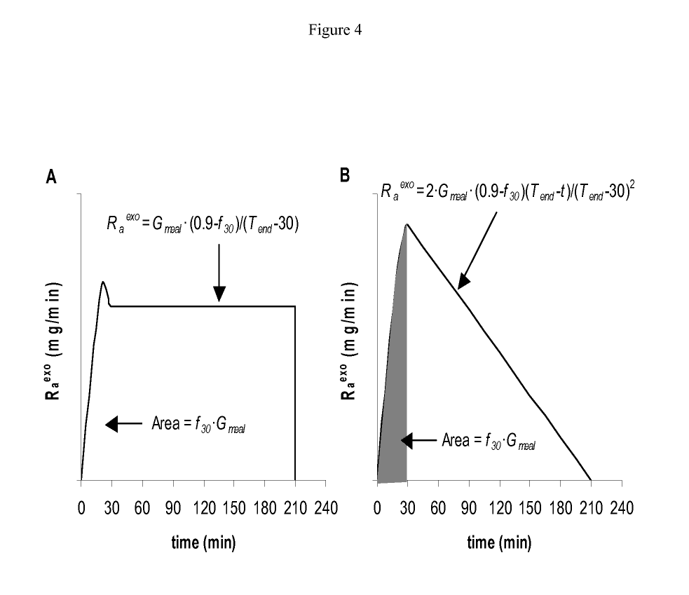

For the amount of glucose absorbed in subsequent time intervals, the profiles described above are approximated by making the following assumptions, which are illustrated in FIG. 4.

Constant absorption assumption assume that the rate of absorption of glucose between t=30 and t=T_{end} is constant and that by t=T_{end}, 90% of the ingested glucose has been absorbed. Thus, between t=30 and t=T_{end}, Rₐ^{exo}(t)=Gₘₑₐₗ (0.9−f₃₀)/(T_{end}−30), where Gₘₑₐₗ is the amount of glucose in the meal, in mg.

Decreasing absorption assumption Here assume that the rate of glucose absorption between t=30 and t=T_{end} is linearly decreasing and goes to 0 at t=T_{end}. A linearly decreasing profile was chosen rather than an exponential decrease for simplicity. Thus, between t=30 and t=T_{end}, Rₐ^{exo}(t)=2Gₘₑₐₗ (0.9−f₃₀) (T_{end}−t)/(T_{end}−30)².

### Using Integral Approach to Determine Parameter Values

The approach of integrating the minimal model equations has been used previously in critical care settings with continuous glucose monitors where the rate of appearance of glucose was known. Hann et al. (2005); and Chase et al. (2006). Here, a similar method is developed in cases where the rate of glucose appearance is unknown. In the derivation that follows, assume that the first two time points at which measurements are taken are at t=0 and t=30 minutes, although this can be easily modified.

Let t₁, t₂, . . . , tₙ, be the time points where glucose and insulin are measured (where, as described above, using t₁=0 and t₂=30 for illustration). Then, integrating both sides of Equation 1 between two time points tⱼ and tₖ gives

$$G ( {t}_{k} ) - G ( {t}_{j} ) = \frac{1}{{V}_{G}} {\int }_{{t}_{j}}^{{t}_{k}} {R}_{a}^{exo} ( t ) d t - {S}_{I} {\int }_{{t}_{j}}^{{t}_{k}} ( G ( t ) \cdot {I}_{i} ( t ) - {G}_{basal} \cdot {I}_{basal} ) d t \qquad ( A6 )$$

The first integral in Equation A6 can be easily integrated for the two profiles described above. In both cases

$${\int }_{0}^{30} {R}_{a}^{exo} ( t ) d t = {f}_{30} \cdot {G}_{meal} \qquad ( A7 )$$

by definition of f₃₀. For the other intervals,

$${\int }_{{t}_{j}}^{{t}_{k}} {R}_{a}^{exo} ( t ) d t = {w}_{jk} ( 0.9 - {f}_{30} ) \cdot {G}_{meal} \qquad ( A8 )$$

where

wⱼₖ=(tₖ−tⱼ)/(T_{end}−30) for constant absorption assumption

wⱼₖ=((T_{end}−tⱼ)²−(T_{end}−tₖ)²)/(T_{end}−30)² for decreasing absorption assumption

To compute the second integral in Equation A6, Equation 2 is first solved for Iᵢ(t) based on the measured plasma insulin concentrations. To do this, Iₚₗₐₛₘₐ(t) is defined by linearly interpolating between the measured time points, i.e., for tⱼ<t<tₖ

$${I}_{plasma} ( t ) = {I}_{plasma} ( {t}_{j} ) + \frac{{I}_{plasma} ( {t}_{k} ) - {I}_{plasma} ( {t}_{j} )}{{t}_{k} - {t}_{j}} ( t - {t}_{j} )$$

Then, Equation 2 can be solved over each interval where tⱼ≦t≦tₖ to give

Iᵢ(t)=αⱼₖ+βⱼₖe^{-(t-t}^{ⱼ})/τ+m_{I}_{ⱼₖ}(t−tⱼ) (A9)

where
m_{I}_{ⱼₖ}=(Iₚₗₐₛₘₐ(tₖ)−Iₚₗₐₛₘₐ(tⱼ))/(tₖ−tⱼ)
δIⱼ=Iᵢ(tⱼ)−Iₚₗₐₛₘₐ(tⱼ)
αⱼₖ=Iₚₗₐₛₘₐ(tⱼ)−τ·m_{I}_{ⱼₖ}
βⱼₖ=δIⱼ+τ·m_{I}_{ⱼₖ}

To obtain Iᵢ(t) over the interval tⱼ≦t≦tₖ using Equation A9, Iᵢ(tⱼ) must be known. This is accomplished in the first interval (where t₁=0 and t₂=30)) by setting Iᵢ(0)=Iₚₗₐₛₘₐ(0)=I_{basal}. In subsequent intervals, Iᵢ(tⱼ) is obtained from Equation A9 over the previous interval tᵢ≦t≦tⱼ.

Now, the second integral in Equation A6 is computed by substituting Iᵢ(t) from Equation A9 and linearly interpolating glucose between the measured points as was done for insulin. Doing this yields

$${Φ}_{jk} = {\int }_{{t}_{j}}^{{t}_{k}} ( G ( t ) {I}_{i} ( t ) - {G}_{basal} {I}_{basal} ) d t = δ {t}_{jk} ( {α}_{jk} G ( {t}_{j} ) - {G}_{basal} {I}_{basal} ) + ( {α}_{jk} {m}_{{G}_{jk}} + {m}_{{I}_{jk}} G ( {t}_{j} ) ) \frac{{( δ {t}_{jk} )}^{2}}{2} + {m}_{{I}_{jk}} {m}_{{G}_{jk}} \frac{{( δ {t}_{jk} )}^{3}}{3} + τ {β}_{jk} G ( {t}_{j} ) ( 1 - {e}^{- δ {t}_{jk} / τ} ) + {τ}^{2} {β}_{jk} {m}_{{G}_{jk}} ( 1 - {e}^{- δ {t}_{jk} / τ} ( 1 + \frac{δ {t}_{jk}}{τ} ) ) \qquad ( A10 )$$

where m_{G}_{ⱼₖ}(G(tₖ)−G(tⱼ))/(tₖ−tⱼ) and δtⱼₖ=tₖ−tⱼ Equation 3 in the main text is obtained by applying equation A6 over each of the intervals, using the results shown in Equations A7-A10, and defining aⱼₖ=−wⱼₖGₘₑₐₗ/V_{G}. In cases where there are additional measurements taken between t=0 and t=30, Equation 3 is still used and Φ₁₂ is computed by summing up the integrals over the subintervals between 0 and 30.

The values of S_{I} and f₃₀ that minimize the squared error in Equation 3 are given by (see, e.g., (Mirsky (1972)))

$$( \begin{matrix}{S}_{I} \\ {f}_{30}\end{matrix} ) = {( {A}^{T} A )}^{- 1} {A}^{T} b$$

where

$$A = ( \begin{matrix}- {Φ}_{12} & {G}_{meal} \\ - {Φ}_{23} & {a}_{23} \\ - {Φ}_{34} & {a}_{34} \\ ⋮ & ⋮\end{matrix} ) , b = ( \begin{matrix}G ( {t}_{2} ) - G ( {t}_{1} ) \\ G ( {t}_{3} ) - G ( {t}_{2} ) + 0.9 {a}_{23} \\ G ( {t}_{4} ) - G ( {t}_{3} ) + 0.9 {a}_{34} \\ ⋮\end{matrix} ) ,$$

and ^{T} and ⁻¹ denote matrix transpose and inverse, respectively.

### Using Minimal Model

The same approach can be applied using the classical minimal model. In this case, Equation 3 in the main text is replaced by

$$( \begin{matrix}G ( {t}_{2} ) - G ( {t}_{1} ) \\ G ( {t}_{3} ) - G ( {t}_{2} ) + 0.9 {a}_{23} \\ G ( {t}_{4} ) - G ( {t}_{3} ) + 0.9 {a}_{34} \\ ⋮\end{matrix} ) = ( \begin{matrix}- {Φ}_{12} & - {Θ}_{12} & {G}_{meal} \\ - {Φ}_{23} & - {Θ}_{23} & {a}_{23} \\ - {Φ}_{34} & - {Θ}_{34} & {a}_{34} \\ ⋮ & ⋮ & ⋮\end{matrix} ) ( \begin{matrix}{S}_{I} \\ GEZI \\ {f}_{30}\end{matrix} ) \qquad ( A11 )$$

where

$$GEZI = {S}_{G} - {S}_{I} \cdot {I}_{basal} and {Θ}_{jk} = {\int }_{{t}_{j}}^{{t}_{k}} ( G ( t ) - {G}_{basal} ) d t$$

is computed in a similar fashion as the integrals described above.

### Parameter Values Used in Examples

1. In all analyses performed, V_{G}=1.5 dl/kg and τ=60 min were used. In initial tests, several different values of τ were tested and τ=60 provided the best overall fit to the data. The value of V_{G} is consistent with what has been reported in other studies and the value of τ is similar to median values that have been reported for 1/P₂ in the classical minimal model. Dalla Man et al. (2004).

The value of T_{end} was obtained from a standard one-dimensional optimization algorithm. For each value of T_{end}, the least squares solution that provided the optimal values of S_{I} and f₃₀ was obtained as described earlier. The value of T_{end} that gave the best fit between the model and experimental data (as judged by r²) was selected. The Matlab® function fminbnd was used to solve the optimization problem, and the value was constrained to be between 150 and 270 min.

### Sensitivity of the Parameter Estimates to Absorption Assumptions

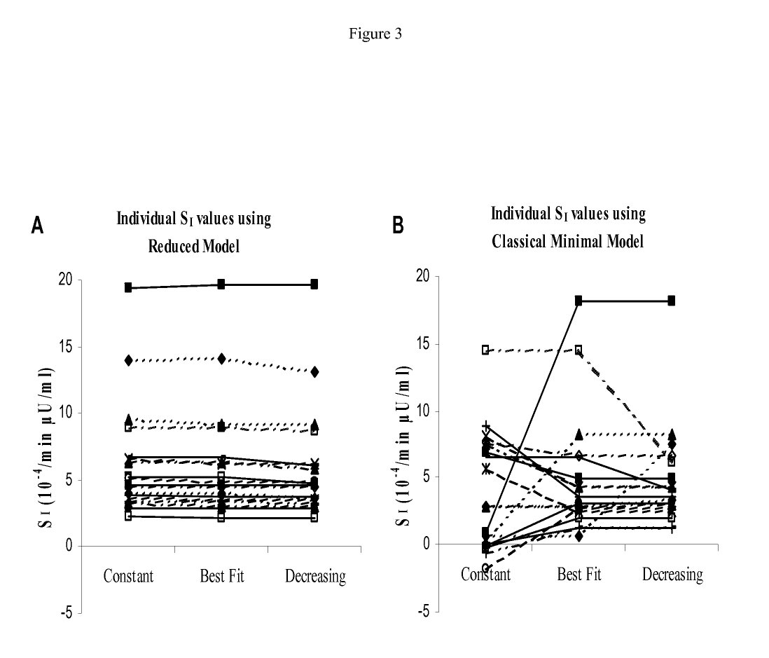

The analysis assumes that the glucose absorption profile can be approximated by one of the two shapes illustrated in FIG. 3. As both of these are approximations to whatever the true profile is, it is important to assess how sensitive the results are to which profile is assumed. In the earlier analyses, the absorption profile that provided the best fit to the data was selected for each subject. Here, results are compared for three different assumptions for the glucose absorption profile: (1) “Constant”: using the constant absorption profile, (2) “Decreasing”: using the linearly decreasing absorption profile, and (3) “Best fit”: using whichever of these two profiles obtains the best fit. This analysis was done using both the full data set containing 11 blood samples and the reduced data consisting of 5 blood samples over 2 hours. In addition, to compare between the reduced model proposed here and the classical minimal model, the analysis was also done using the parameter identification approach with the classical minimal model. The results using the reduced model are insensitive to the absorption assumptions, whereas results using the classical minimal model are more sensitive to these assumptions, as illustrated in Table 3 and FIG. 3. When using the classical minimal model, the best fit is occasionally achieved with a negative value of S_{I} (a good fit can sometimes be achieved with a relatively large S_{G} and a negative S_{I}). The average variation in the S_{I} values obtained with the different absorption assumptions was less than 5% when using the reduced model and greater than 150% when using the classical minimal model. In Table 3, the mean values (+s.e.m.) for S_{I} (10⁻⁴/min/(μU/ml)). S_{I} for each subject was computed 6 different ways as described in the text. The results for the reduced model are less sensitive to the absorption assumptions than those obtained using the classical minimal model. In addition, the reduced model produces good results when using only 5 data points and the classical minimal model does not.

TABLE 3

| Absorption | Reduced model | | | Classical minimal model | | |
|---|---|---|---|---|---|---|
| assumption | Best fit | Constant | Decreasing | Best fit | Constant | Decreasing |
| Using 11 | 6.3 ± 1.0 | 6.3 ± 1.0 | 6.1 ± 1.1 | 6.3 ± 1.0 | 3.9 ± 1.0 | 4.8 ± 0.9 |
| data points | | | | | | |
| Using 5 | 6.2 ± 1.1 | 6.2 ± 1.1 | 5.9 ± 1.0 | −2.2 ± 2.0 | 3.6 ± 1.2 | −4.8 ± 1.8 |
| data points | | | | | | |

### Akaike Information Criteria Prefers Reduced Model Over Classical Minimal Model

The Akaike information criteria (AIC), which was developed based on information theory (Akaike (1974)), is a frequently used statistical measure to assess the goodness-of-fit of models to data. The criteria seeks to determine an optimal tradeoff between goodness-of-fit of the model to experimental data and complexity of the model. Due to the relatively small number of measurements, AICc was used. Schirra et al. (1996).

AICc suggests that the reduced model of glucose metabolism should be preferred over the classical minimal model for OGTTs, particularly when few data points are collected. When using only 5 data points, the reduced model was preferred for all subjects in both studies. In Study 2, the reduced model was preferred in over 90% of the subjects when 6 data points were used (t=0, 30, 60, 90, 120, and 180) and over 70% of the subjects when all 11 data points were used.

### Discussion

There is considerable interest in obtaining a robust and practical method for determining insulin sensitivity under physiologic conditions. The present method is convenient both experimentally and analytically and can therefore be used in large-scale clinical trials. As such, the method can be performed using an OGTT or mixed meal with as few as about 5 blood samples over about a 2-hour period. The assumptions associated with the method are valid both in untreated subjects and in subjects treated with various pharmaceutic therapies (including therapies that modify insulin secretion, insulin sensitivity, and/or the rate of glucose absorption). In addition, the method that produces results that are highly correlated with results from hyperinsulinemic clamp studies. Finally, to facilitate the adoption of the method, the results are easily obtained without requiring specialized software.

Several methods have been proposed in recent years for estimating insulin sensitivity from OGTTs. e.g., Dalla Man et al. (2005a); Dalla Man et al. (2005b); Caumo et al. (2000); Stumvoll et al. (2000); Matsuda et al. (1999); Hansen et al. (2007); and Mari et al. (2001). Although progress has been made with these methods, none of the previously developed methods fully satisfied the above criteria. The empiric methods were derived using untreated subjects and, as such, it is unclear how well these relationships will hold up for a variety of different therapies. The OGIS method (Mari et al. (2001)) has the benefit of only requiring 3 blood samples and allows insulin sensitivity to be determined from an algebraic formula. However, there are several limitations associated with this method. First, the method assumes that every individual has the same rate of glucose absorption at the instant t=90 or t=120 min. This assumption is questionable in untreated subjects and even less likely to be valid for treatments such as acarbose or exenatide that alter the rate of glucose absorption. Second, by attempting to fit the ordinary differential equations describing glucose kinetics at a single time point, much of the information available in the data is ignored. Finally, even though the OGIS method contained a number of parameters that were fit specifically to match data from hyperinsulinemic clamps, correlations with independent clamp data are not very high. The value of r²=0.55 is even higher than what was reported using other data (Akaike (1974)), yet this correlation is comparable to what is obtained with QUICKI, which requires only a single blood sample.

Caumo's method (2000) assumes that the rate of glucose absorption closely matches the plasma glucose curve. While this approximation seems reasonable in untreated subjects, therapies such as insulin secretion enhancers and/or insulin sensitizers can alter the plasma glucose concentrations without changing the rate of glucose absorption. Therefore, this assumption may not be valid when comparing treated and untreated responses. In addition, the derivation of the equation requires measurements taken sufficiently long after a meal so that both glucose and insulin action are back to baseline values, meaning that this method requires data to be collected for at least 3 hours after an OGTT. The method proposed by Dalla Man et al. (2005a) has reported promising results in untreated subjects, but it requires at least 7 blood samples and the method requires identifying as many parameters as data points collected. In addition, this method requires special modeling software, requires prior distributions to be defined for parameters to improve their identifiability, and there is no guarantee of finding a unique optimal solution. A comparison of the assumptions regarding glucose absorption for the various methods is shown in Table 4.

TABLE 4

| | | Absorption parameters fit |
|---|---|---|
| Method | Assumptions about glucose absorption | from data |
| S_{I}ARₐA | Shape of glucose absorption profile is | f₃₀ |
| | assumed to be well approximated by | T_{end} |
| | one of the two profiles illustrated in FIG. 4. | Shape of profile that |
| | 90% of the ingested glucose appears | provides best fit |
| | in systemic circulation after an OGTT. | (constant or decreasing) |
| OGIS Hansen | Every individual is assumed to have | None |
| et al. (2007) | the same rate of glucose absorption at | |
| | the instant t = 90 or t = 120 min. | |
| Caumo | Shape of glucose absorption profile is | None |
| (2000) | assumed to resemble an “anticipated | |
| | version” of the plasma glucose | |
| | excursion. | |
| | 80% of the ingested glucose appears | |
| | in systemic circulation after an | |
| | OGTT. | |
| Dalla Man | Absorption is represented as a | A parameter at each |
| (2005a) | piecewise linear function; values at | time where glucose is |
| | each measurement point are identified | measured is identified |
| | from data. | to obtain the piecewise |
| | 90% of the ingested glucose appears | linear representation of |
| | in systemic circulation after an | Rₐ^{exo} |
| | OGTT. | |

The S_{I}ARₐA method provided herein meets all of the criteria for a practical method. In addition to being easy to perform, both experimentally and analytically, the method has been validated in several ways. It was shown to be able to distinguish different patient types, to be able to identify change in insulin sensitivity in response to pharmaceutic therapy using only a small number of subjects, and results from the S_{I}ARₐA method are highly correlated with results from hyperinsulinemic clamps. Importantly, the results were shown to be relatively insensitive to different assumptions that were made in the analysis.

Although this analysis was focused on determining insulin sensitivity from OGTTs, there are several other potential applications. Because the method identifies the amount of glucose absorbed in the first 30 minutes and provides information in the shape of the absorption profile after that, it has potential to be useful in diagnosing diabetic gastroparesis or other situations involving inappropriate gastric emptying. Another potential application is to apply the integral method for parameter identification approach to FSIVGTT data. While the reduced model has several advantages when using the S_{I}ARₐA method with OGTT data, the classical minimal model is likely to be preferred when using this method with FSIVGTT data (the additional glucose effectiveness parameter in the classical minimal model helps fit the glucose response following rapid IV glucose injection).

Two additional areas of suggested future research are further validating the glucose absorption estimates obtained by this method and validating this method for mixed meal challenges. The identified f₃₀ values are consistent with data reported in (Dalla Man et al. (2005a)) using tracers. Because nothing in the derivation of the method was specific for OGTTs, it is expected that the method should also be applicable for mixed meal tests. Tests of the method using data from mixed meals provided excellent agreement between the model and the experimental data (r²>0.95).

As with any approach of applying mathematical modeling to analyzing experimental data, there were several assumptions made in the analysis. The main assumptions made here were that (1) the net rate of glucose disposal following a meal can be reasonably approximated by S_{I}(G·I−G_{basal}·I_{basal}), (2) the shape of the glucose absorption profile after 30 minutes can be reasonably approximated by one of the 2 profiles described, and (3) good results could be obtained when the distribution volume of glucose and the time constant associated with insulin transfer into interstitial fluid were specified rather than fit from the data. The excellent fit between the model and the data (r²˜0.95) suggests that assumption (1) is valid for the applications tested so far and the sensitivity analyses performed and the Akaike Information Criteria suggest that this approximation is preferred over the classical minimal model in these applications. In addition, the method allows other models (including the classical minimal model) to be tested if desired. Assumption (2) provides approximations that are consistent with gastric emptying and glucose absorption data; other profiles can also be tested by specifying different values of the wᵢⱼ parameters. Finally, in order to obtain the most robust estimates for the parameters of interest from a limited number of data points, V_{G} and τ were specified rather than to attempt to fit these from the data. The method provides an overall measure-of-fit and uncertainty estimates associated with the parameter values that can be used to assess whether there are cases where these assumptions are not appropriate.

In summary, the present method determines insulin sensitivity and glucose absorption during an OGTT or mixed meal. The S_{I}ARₐA method consists of a reduced mathematical model of glucose kinetics, a novel method for approximating glucose absorption during a meal or OGTT, and an integral approach that allows the parameters to be determined using an algebraic formula that can easily be implemented in a standard spreadsheet. The method has been shown to be able to distinguish different patient types, to identify changes in insulin sensitivity arising from pharmaceutic therapy, and results from the S_{I}ARₐA method are highly correlated with results from hyperinsulinemic clamps. This method provides a convenient and robust means for assessing insulin sensitivity under physiologic conditions.

### REFERENCES

Akaike (1974) A new look at statistical model identification. IEEE Transactions on Automatic Control 19:716-723
Allen et al. (2006) Halofenate is a selective peroxisome proliferator-activated receptor gamma modulator with antidiabetic activity Diabetes 55:2523-33
American Diabetes Association (1996) Self-monitoring of blood glucose consensus statement Diabetes Care 19(Suppl 1):S62-6
Bergman (1989) Lilly lecture 1989. Toward physiological understanding of glucose tolerance. Minimal-model approach Diabetes 38:1512-1527
Brener et al. (1983) Regulation of the gastric emptying of glucose Gastroenterol 85:76-82
Brun et al. (2000) Homeostasis model assessment and related simplified evaluations of insulin sensitivity from fasting insulin and glucose Diabetes Care 23:1037-1038
Burkey et al. (2005) Acute and chronic effects of the incretin enhancer vildagliptin in insulin-resistant rats J Pharmacol Exp Ther 315:688-95
Carey et al. (2002) Effect of Rosiglitazone on insulin sensitivity and body composition in type 2 diabetic patients Obes Res 10:1008-1015
Caumo et al. (2000) Insulin sensitivity from meal tolerance tests in normal subjects: a minimal model index J Clin Endocrinol Metab 85:4396-4402
Chase et al. (2006) Integral-based filtering of continuous glucose sensor measurements for glycaemic control in critical care Comput Methods Programs Biomed 82:238-247
Chiasson et al. (1996) The effect of acarbose on insulin sensitivity in subjects with impaired glucose tolerance Diabetes Care 19:1190-1193
Cutfield et al. (2003) Evaluation of HOMA and QUICKI as measures of insulin sensitivity in prepubertal children Pediatr Diabetes 4:119-125
Dalla Man et al. (2004) Minimal model estimation of glucose absorption and insulin sensitivity from oral test: validation with a tracer method Am J Physiol Endocrinol Metab 287:637-643
Dalla Man et al. (2005a) Two-hour seven sample oral glucose tolerance test and meal protocol: minimal model assessment of beta-cell responsivity and insulin sensitivity in nondiabetic individuals Diabetes 54:3265-3273
Dalla Man et al. (2005b) Insulin sensitivity by oral glucose minimal models: validation against clamp Am J Physiol Endocrinol Metab 289:954-959
DeFronzo et al. (1979) Glucose clamp technique: a method for quantifying insulin secretion and resistance Am J Physiol 237:E214-223
Delgado et al. (2002) Acarbose improves indirectly both insulin resistance and secretion in obese type 2 diabetic patients Diabetes Metab 28:195-200
Emoto et al. (1999) Homeostasis model assessment as a clinical index of insulin resistance in type 2 diabetic patients treated with sulfonylureas Diabetes Care 22:818-822
Gastaldelli et al. (2007) Thiazolidinediones improve P-cell function in type 2 diabetic patients Am J Physiol Endocrinol Metab 292:E871-83
Hann et al. (2005) Integral-based parameter identification for long-term dynamic verification of a glucose-insulin system model Comput Methods Programs Biomed 77:259-270
Hansen et al. (2007) The BIGTT Test: A novel test for simultaneous measurement of pancreatic β-cell function, insulin sensitivity, and glucose tolerance Diabetes Care 30:257-262
Hatanaka et al. (1994) The measurement of gastric emptying in conscious rats by monitoring serial changes in serum acetaminophen level J Pharmacol Toxicol Methods 31:161-5
Hsieh et al. (2007) Both slow-release and regular-form metformin improve glycemic control without altering plasma visfatin level in patients with type 2 diabetes mellitus Metabolism 56:1087-92
Hunt et al. (1985) Effect of meal volume and energy density on the gastric emptying of carbohydrates Gastroenterol 89:1326-1330
Katz et al. (2000) Quantitative insulin sensitivity check index: a simple, accurate method for assessing insulin sensitivity in humans J Clin Endocrinol Metab 85:2402-2410
Livesey et al. (1998) Simultaneous time-varying systemic appearance of oral and hepatic glucose in adults monitored with stable isotopes Am J Physiol 275(4 Pt 1):717-728
Mari et al. (2001) A model-based method for assessing insulin sensitivity from the oral glucose tolerance test Diabetes Care 24:539-548
Mari et al. (2005) Vildagliptin, a Dipeptidyl Peptidase-IV Inhibitor, Improves Model-Assessed β-Cell Function in Patients with Type 2 Diabetes JCEM 90:4888-4894
Matsuda et al. (1999) Insulin sensitivity indices obtained from oral glucose tolerance testing: comparison with the euglycemic insulin clamp Diabetes Care 22:1462-1470
Matthews et al. (1985) Homeostasis model assessment: insulin resistance and beta-cell function from fasting plasma glucose and insulin concentrations in man Diabetologia 28:412-419
Mayerson et al. (2002) The effects of Rosiglitazone on insulin sensitivity, lipolysis, and hepatic and skeletal muscle triglyceride content in patients with type 2 diabetes Diabetes 51:797-802
Medhus et al. (2001) Gastric emptying: the validity of the paracetamol absorption test adjusted for individual pharmacokinetics Neurogastroenterol Mot 13:179-85
Meneilly et al. (2000) Effect of acarbose on insulin sensitivity in elderly patients with diabetes Diabetes Care 23:1162-7
Mokdad et al. (2003) Prevalence of obesity, diabetes, and obesity-related health risk factors, 2001 JAMA 289:76-79
Natalucci et al. (2003) Age-related analysis of glucose metabolism in spontaneously hypertensive and normotensive rats Exp Physiol 88:399-404
Ovalle et al. (2004) Effect of rosiglitazone versus insulin on the pancreatic β-cell function of subjects with type 2 diabetes Diabetes Care 27:2585-2589
Polidori et al. (2008) S_{I}ARₐA method for determining insulin sensitivity and glucose absorption Part 2: Application in Animals Highlights Disparate Response to Therapies
Polidori (2008) S_{I}ARₐA: A Simple Method for Determining Insulin Sensitivity and Glucose Absorption Rates from Oral Glucose Tolerance Tests. Part 1: Validation in Humans
Rasouli et al. (2007) Effects of pioglitazone and metformin on β-cell function in nondiabetic subjects at high risk for type 2 diabetes Am J Physiol Endocrinol Metab 292:E359-65
Reaven (1997) Banting Lecture 1988. Role of insulin resistance in human disease 1988 Nutrition 13:65
Richardson et al. (2006) Effects of rosiglitazone and metformin on pancreatic beta cell gene expression Diabetologia 49:685-96
Rivers et al. (2006) Precision and Accuracy of Two Blood Glucose Meters: FreeStyle Flash Versus One Touch Ultra Am J Health-Syst Pharm 63:1411-1416
Rose et al. (2001) Effect of prior exercise on glucose metabolism in trained men Am J Physiol Endocrinol Metab 281:E766-71
Sakamoto et al. (1997) The accuracy of glucose effectiveness at zero insulin with reduced sample protocol from the minimal model Med Sci Sports Ex 29:20
Schirra et al. (1996) Gastric emptying and release of incretin hormones after glucose ingestion in humans J Clin Invest 97:92-103
Seltzer et al. (1967) Insulin secretion in response to glycemic stimulus: relation of delayed initial release to carbohydrate intolerance in mild diabetes mellitus J Clin Invest 46:323-35
Stumvoll et al. (2000) Use of the oral glucose tolerance test to assess insulin release and insulin sensitivity Diabetes Care 23:295-301
van Doorn et al. (2006) Evaluation of proinflammatory cytokines and inflammation markers as biomarkers for the action of thiazolidinediones in Type 2 diabetes mellitus patients and healthy volunteers Br J Clin Pharmacol 62:391-402
van Doorn et al. (2007) Evaluation of metabolite profiles as biomarkers for the pharmacological effects of thiazolidinediones in Type 2 diabetes mellitus patients and healthy volunteers Br J Clin Pharmacol 63:562-74
Vella et al. (2007) Effects of dipeptidyl peptidase-4 inhibition on gastrointestinal function, meal appearance, and glucose metabolism in type 2 diabetes Diabetes 56:1475-80
Vidon et al. (1988) Metformin in the digestive tract Diabetes Res Clin Pract 4:223-9
Wachters-Hagedoom et al. (2006) The rate of intestinal glucose absorption is correlated with plasma glucose-dependent insulinotropic polypeptide concentrations in healthy men J Nutr 136:1511-6
Weitgasser et al. (1999) Newer portable glucose meters—analytical improvement compared with previous generation devices? Clin Chem 45:1821-5
Yasuda et al. (2004) Metformin causes reduction of food intake and body weight gain and improvement of glucose intolerance in combination with dipeptidyl peptidase IV inhibitor in Zucker fa/fa rats J Pharmacol Exp Ther 310:614-9
Yokoyama et al. (2003) Quantitative insulin sensitivity check index and the reciprocal index of homeostasis model assessment in normal range weight and moderately obese type 2 diabetic patients Diabetes Care 26:2426-2432

1. A method of determining insulin sensitivity from an oral glucose tolerance test or mixed meals comprising the steps of
measuring blood glucose and insulin levels after a meal
analyzing the measurements using
a. the differential equations (1) and (2) shown below
b. a method for approximating the rate of appearance of glucose from the meal using at least 1-2 parameters, as illustrated in FIG. 4
c. the integral approach described in the present application enabling the parameters to be obtained by solving the algebraic equation shown in Equation 3
$$\begin{matrix}\frac{d G}{d t} = \frac{{R}_{a}^{exo} ( t )}{{V}_{G}} - {S}_{I} ( G \cdot {I}_{i} - {G}_{basal} \cdot {I}_{basal} ) & ( 1 ) \\ \frac{d {I}_{i}}{d t} = \frac{1}{τ} ( {I}_{plasma} - {I}_{i} ) & ( 2 )\end{matrix}$$
where
G(t) is the plasma glucose concentration in mg/dl
Rₐ^{exo}(t) is the rate of appearance of exogenous glucose into the plasma (from meals or injections/infusions) in mg/min
V_{G} is the distribution volume of glucose in dl
S_{I} is insulin sensitivity in 1/min/(μU/ml)
Iᵢ(t) is the interstitial insulin concentration, in μU/ml (In addition to a time delay between plasma and interstitial insulin concentrations, interstitial concentrations are also lower than plasma concentrations, even in steady state conditions; this difference is not accounted for in these equations. Thus, the proper interpretation of Iᵢ(t) is as the actual interstitial insulin concentration at time t multiplied by the ratio of basal plasma insulin/basal interstitial insulin)
G_{basal} is the basal plasma glucose concentration in mg/dl
I_{basal} is the basal plasma insulin concentration μU/ml
τ is the time constant associated with transfer of insulin from plasma to interstitial fluid, in min
Iₚₗₐₛₘₐ is the plasma insulin concentration, in μU/ml.
$$( \begin{matrix}G ( {t}_{2} ) - G ( {t}_{1} ) \\ G ( {t}_{3} ) - G ( {t}_{2} ) + 0.9 {a}_{23} \\ G ( {t}_{4} ) - G ( {t}_{3} ) + 0.9 {a}_{34} \\ ⋮\end{matrix} ) = ( \begin{matrix}- {Φ}_{12} & {G}_{meal} / {V}_{G} \\ - {Φ}_{23} & {a}_{23} \\ - {Φ}_{34} & {a}_{34} \\ ⋮ & ⋮\end{matrix} ) ( \begin{matrix}{S}_{I} \\ {f}_{30}\end{matrix} ) \qquad ( 3 )$$
where
t₁, t₂, . . . , tₙ are the time points where glucose and insulin are measured
G(t₁), G(t₂), . . . are the plasma glucose values at t₁, t₂, . . . Gₘₑₐₗ is the total amount of glucose consumed in the meal
a₂₃, a₃₄, . . . are obtained based on the approximate absorption profiles as described
Φ₁₂, Φ₂₃, . . . are obtained based on the plasma glucose and insulin measurements as described

2. The method according to claim 1 wherein the results are obtained from at least about four to six samples.

3. The method according to claim 1 wherein the results are obtained during a period of about two to four hours.

4. The method according to claim 1 wherein the method is used to determine the effect of therapy on insulin sensitivity.

5. The method according to claim 4 wherein the therapy is pharmaceutical, nutritional or behavioral.

6. The method according to claim 1 wherein the method is used in preclinical studies to determine the effect of a therapy.

7. The method according to claim 1 wherein the method is used as a prognostic to assess a patient's risk of developing a disease or syndrome.

8. The method according to claim 7 wherein the disease or syndrome is diabetes or metabolic syndrome.

9. The method according to claim 1 wherein the method is used to monitor and/or adjust patient treatment.

10. The method according to claim 1 wherein the method is used in conjunction with automated insulin delivery.
